(19) United States
(12) Patent Application Publication
(10) Pub. No.: US 2007/0055263 A1
(43) Pub. Date: Mar. 8, 2007

(54) Tools for Percutaneous Spinal Ligament Decompression and Device for Supporting Same

(75) Inventors: Bryce Way, San Jose, CA (US); Donald Schomer, Englewood, CO (US); Murray David Solsberg, Englewood, CO (US)

Correspondence Address: CONLEY ROSE, P.C., P. O. BOX 3267, HOUSTON, TX, 77253-3267, US

(73) Assignee: X-Sten Corp., Englewood, CO (US)

(21) Appl. No.: 11/461,020

(22) Filed: Jul. 31, 2006

Related U.S. Application Data
(60) Provisional application No. 60/703,921, filed on Jul. 29, 2005.
(60) Provisional application No. 60/733,819, filed on Nov. 4, 2005.

(51) Int. Cl. A61B 17/00

(52) U.S. Cl. 606/82

(57) ABSTRACT

A device for providing percutaneous access to a surgical site. In an embodiment, the device comprises a handle. In addition, the device comprises a bone-cutting member extending from the handle, wherein the bone-cutting member includes a handle end fixed to the handle and a cutting end. Further, the device comprises a portal including a first end, a second end, and a through bore extending therebetween, wherein the bone-cutting member is disposed within the through bore and concentric with the portal Still further, the portal has a first position with the second end releasably coupled to the handle and a second position with the second end released from the handle and the bone-cutting member.

### CROSS-REFERENCE TO RELATED APPLICATIONS

This application claims benefit of U.S. provisional application Ser. No., 60/703,921 filed Jul. 29, 2005, and entitled “Tools for Percutaneous Spinal Ligament Decompression and Device for Supporting Same,” which is hereby incorporated herein by reference in its entirety. This application also claims benefit of U.S. provisional application Ser. No. 60/733,819 filed Nov. 4, 2005, and entitled “Bone Wax Delivery Device,” which is hereby incorporated herein by reference in its entirety.

### STATEMENT REGARDING FEDERALLY SPONSORED RESEARCH OR DEVELOPMENT

Not applicable.

### BACKGROUND

1. Field of the Invention

The present invention relates generally to a minimally invasive method, device and system for treating spinal disorders using imaging guidance. More particularly, this invention relates to devices and tools that provide a percutaneous portal to tissues in a region of interest. Still more particularly, this invention relates to devices and tools that provide percutaneous portals to tissue through bone.

2. Background of the Invention

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae with an intervertebral disc between adjacent vertebrae The vertebrae are stabilized by muscles and ligaments that hold the vertebrae in place and limit the movements of the vertebrae.

As illustrated in FIG. 1, each vertebra 10 includes a vertebral body 12 that supports a vertebral arch 14. A median plane 210 generally divides vertebra 10 into two substantially equal lateral sides. Vertical body 12 has the general shape of a short cylinder and is anterior to the vertebral arch 14. The vertebral arch 14 together with vertebral body 12 encloses a space termed the vertebral foramen 15. The succession of vertebral foramen 15 in adjacent vertebrae 10 along the vertebral column define the vertebral canal (spinal canal), which contains the spinal cord.

Vertebral arch 14 is formed by two pedicles 24 which project posteriorly to meet two laminae 16. The two laminae 16 meet posteriomedially to form the spinous process 18. At the junction of pedicles 24 and laminae 16, six processes arise. Two transverse processes 20 project posterolaterally, two superior articular processes 22 project generally superiorly and are positioned superior to two inferior articular processes 25 that generally project inferiorly.

The vertebral foramen 15 is generally an oval shaped space that contains and protects the spinal cord 28. Spinal cord 28 comprises a plurality of nerves 34 surrounded by cerebrospinal fluid (CSF) and an outermost sheath/membrane called the dural sac 32. The CSF filled dural sac 32 containing nerves 34 is relatively compressible. Posterior to the spinal cord 28 within vertebral foramen 15 is the ligamentum flavum 26. Laminae 16 of adjacent vertebral arches 14 in the vertebral column are joined by the relatively broad, elastic ligamentism flavum 26.

In degenerative conditions of the spine, narrowing of the spinal canal (stenosis) can occurs. Lumbar spinal stenosis is often defined as a dural sac cross-sectional area less than 100 mm₂ or an anteroposterior (AP) dimension of the canal of less than 10-12 mm for an average male.

The source of many cases of lumbar spinal stenosis is thickening of the ligamentum flavum, Spinal stenosis may also be caused by subluxation, facet joint hypertrophy, osteophyte formation, underdevelopment of spinal canal, spondylosis deformians, degenerative intervertebral discs, degenerative spondylolisthesis, degenerative arthritis, ossification of the vertebral accessory ligaments and the like. A less common cause of spinal stenosis, which usually affects patients with morbid obesity or patients on oral corticosteroids, is excess fat in the epidural space. The excessive epidural fat compresses the dural sac, nerve roots and blood vessels contained therein and resulting in back and leg pain and weakness and numbness of the legs. Spinal stenosis may also affect the cervical and, less commonly, the thoracic spine.

Patients suffering from spinal stenosis are typically first treated with exercise therapy, analgesics and anti-inflammatory medications. These conservative treatment options frequently fail. If symptoms are severe, surgery is required to decompress the canal and nerve roots.

In some conventional approaches to correct stenosis in the lumbar region, an incision is made in the back and the muscles and supporting structures are stripped away from the spine, exposing the posterior aspect of the vertebral column. The thickened ligamentum flavum is then exposed by removal of a portion of the vertebral arch, often at the laminae, covering the back of the spinal canal (laminectomy). The thickened ligamentum flavum ligament can then be excised by sharp dissection with a scalpel or punching instruments such as a Kerison punch that is used to remove small chips of tissue. The procedure is performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently.

Much of the pain and disability after an open laminectomy results from the tearing and cutting of the back muscles, blood vessels, supporting ligaments, and nerves that occurs during the exposure of the spinal column. Also, because the spine stabilizing back muscles and ligaments are stripped and detached from the spine during the laminectomy, these patients frequently develop spinal instability post-operatively.

Minimally invasive techniques offer the potential for less post-operative pain and faster recovery compared to traditional open surgery Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures.

Various techniques for minimally invasive treatment of the spine are known. Microdiscectomy is performed by making a small incision in the skin and deep tissues to create a portal to the spine. A microscope is then used to aid in the dissection of the adjacent structures prior to discectomy. The recovery for this procedure is much shorter than traditional open discectomies. Percutaneous discectomy devices with fluoroscopic guidance have been used successfully to treat disorders of the disc but not to treat spinal stenosis or the ligamentum flavum directly. Arthroscopy or direct visualization of the spinal structures using a catheter or optical system have also been proposed to treat disorders of the spine including spinal stenosis, however these devices still use miniaturized standard surgical instruments and direct visualization of the spine similar to open surgical procedure. These devices and techniques are limited by the small size of the canal and these operations are difficult to perform and master. In addition, these procedures are painful and often require general anesthesia. Further, the arthroscopy procedures are time consuming and the fiber optic systems are expensive to purchase and maintain.

Still further, because the nerves of the spinal cord pass through the spinal canal directly adjacent to and anterior to the ligamentum flavum, any surgery, regardless of whether open or percutaneous, includes a risk of damage to the nerves of the spinal cord.

Hence, it remains desirable to provide simple methods, techniques, and devices for treating spinal stenosis and other spinal disorders without requiring open surgery. It is further desired to provide a system whereby the risk of damage to the dural sac containing the spinal nerves may be reduced.

### SUMMARY OF THE INVENTION

These and other needs in the art are addressed in one embodiment by a device for providing percutaneous access to a surgical site. In an embodiment, the device comprises a handle In addition, the device comprises a bone-cutting member extending from the handle, wherein the bone-cutting member includes a handle end fixed to the handle and a cutting end, Further, the device comprises a portal including a first end, a second end, and a through bore. extending therebetween, wherein the bone-cutting member is disposed within the through bore. Still further, the portal has a first position in which the second end is releasably coupled to the handle and a second position in which the second end is released from the handle and the bone-cutting member.

Theses and other needs in the art are addressed in another embodiment by a system for performing a percutaneous ligamentum flavum decompression. In an embodiment, the system comprises a handle. In addition, the system comprises a bone-cutting member extending from the handle. Further, the system comprises a portal including a cannulated member extending from the handle and concentric with the bone-cutting member, wherein the portal is releasably coupled to the handle. Still further, the system comprises a tissue-excision device sized and configured to pass through the portal.

Theses and other needs in the art are addressed in another embodiment by a method for treating stenosis in a spine, the spine including a thecal sac, a spinal canal and an epidural space therebetween, the stenosis determining a region of interest in the spine. In an embodiment, the method comprises the step of compressing the thecal sac in the region of interest by injecting a fluid to form a safety zone and establish a working zone, the safety zone lying between the working zone and the thecal sac. In addition, the method comprises the step of percutaneously cutting a hole through a lamina of the spine adjacent the region of interest. Further, the method comprises the step of positioning a portal through the hole to provide access to the region of interest. Still further, the method comprises the step of inserting a tissue-excision tool through the portal and into tissue in the working zone. Moreover, the method comprises the step of using the tool to percutaneously reduce the stenosis. In addition, the method comprises the step of utilizing imaging to visualize the position of the tool during at least a part of method.

The foregoing has outlined rather broadly the features and technical advantages of embodiments of the present invention in order that the detailed description that follows may be better understood. Additional features and advantages of embodiments of the present invention will be described hereinafter that form the subject of the claims. It should be appreciated by those skilled in the art that the conception and the specific embodiments disclosed may be readily utilized as a basis for modifying or designing other structures for carrying out the same purposes. It should also be realized by those skilled in the art that such equivalent constructions do not depart from and scope of the invention as set forth in the appended claims.

### BRIEF DESCRIPTION OF THE DRAWINGS

For a more complete understanding of the invention, reference is made to the accompanying drawings, wherein:

FIG. 1 is cross-section of the spine viewed from the space between two vertebrae, showing the upper surface of one vertebra and the spinal canal with the dural sac and a normal (un-stenosed) ligamentum flavum therein;

FIG. 2 is an illustration of the same section as FIG. 1, showing the spinal canal with the dural sac and a thickened ligamentum flavum therein;

FIG. 3 is an enlarged cross-section of a vertebral foramen, showing a safety zone created by compression of the dural sac;

FIG. 4 is the cross-section of FIG. 3, showing a tissue excision tool positioned in the ligamentum flavum;

FIGS. 5-9 are a series of illustrations showing tissue excision by a tissue-excision tool constructed in accordance with a first embodiment of the invention;

FIGS. 10-14 are a series of illustrations showing tissue excision by a tissue-excision tool constructed in accordance with a second embodiment of the invention;

FIGS. 15 and 17 are sequential illustrations showing removal of tissue from a tissue-excision tool by a tissue-removal device constructed in accordance with an embodiment of the invention;

FIGS. 16 and 18 are end views of the tissue-removal device of FIGS. 15 and 17, respectively;

FIG. 19 shows an alternative embodiment of a grasping needle with a corkscrew shape;

FIG. 20 is a perspective view of a tissue-excision tool constructed in accordance with a third embodiment of the invention;

FIGS. 21 and 22 are enlarged cross-sectional and perspective views, respectively, of the grasping device of FIG. 20 in its retracted position;

FIGS. 23 and 24 are enlarged cross-sectional and perspective views, respectively, of the grasping device of FIG. 20 in its extended position;

FIG. 25 is a schematic illustration of one embodiment of a double-ended ligament anchor being deployed in a ligamentum flavum;

FIG. 26 shows the device of FIG. 25 after full deployment;

FIGS. 27 is a perspective view of an entire tool constructed in accordance with preferred embodiments;

FIG. 28 is an enlarged cross-sectional view of the distal tip of the tool of FIG. 27 with the aperture partially opened;

FIG. 29 is a cross-sectional view of the handle end of the tool of FIG. 27;

FIG. 30 is cross-sectional view of another embodiment of a tissue-removal device;

FIG. 31 is a side view of a portal-emplacing tool constructed in accordance with one embodiment of the invention;

FIG. 32 is a side view of the handle and bone-cutting member of the portal-emplacing tool of FIG. 31;

FIG. 33 is a side of the portal of the portal-emplacing tool of FIG. 31;

FIG. 34 is a schematic diagram of the portal-emplacing tool of FIG. 31 forming a hole in a lamina;

FIG. 35 is a schematic diagram of the portal of the portal-emplacing tool of FIG. 31 positioned and anchored in a lamina;

FIG. 36 is a schematic diagram of a tissue excision tool accessing an enlarged ligamentum flavum through the portal of FIG. 35;

FIG. 37 is a side elevation of a portal-repositioning tool that may be to reposition the portal of FIG. 31 relative to the lamina; and

FIG. 38 is a side view of multiple embodiments of a bone wax application device.

### DETAILED DESCRIPTION OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment

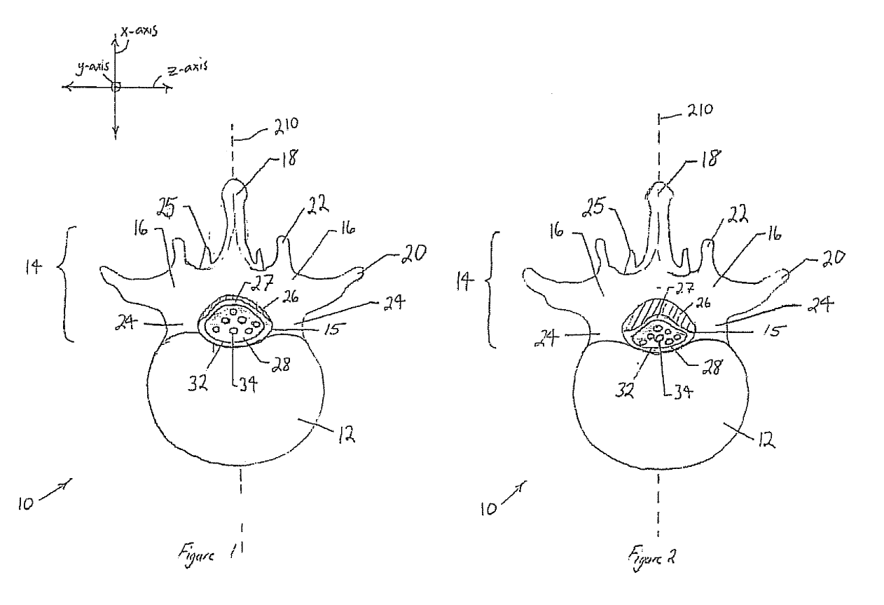

For purposes of this discussion, the x-, y-, and z-axes are shown in FIGS. 1, 3, 5, 6, and 7 to aid in understanding the descriptions that follow. The x-, y-, and z-axes have been assigned as follows. The x-axis is perpendicular to the longitudinal axis of the vertebral column and perpendicular to the coronal/frontal plane (i.e., x-axis defines anterior vs. posterior relationships). The y-axis runs substantially parallel to the vertebral column and perpendicular to the transverse plane (i.e., y-axis defines superior vs. inferior relationships). The z-axis is perpendicular to the longitudinal axis of the vertebral column and perpendicular to the median/midsagittal plane (i.e., z-axis defines the lateral right and left sides of body parts). The set of coordinate axes (x-, y-, and z-axes) are consistently maintained throughout although different views of vertebrae and the spinal column may be presented.

It is to be understood that the median/midsagittal plane passes from the top to the bottom of the body and separates the left and the right sides of the body, and the spine, into substantially equal halves (e.g., two substantially equal lateral sides). Further, it is to be understood that the frontal/coronal plane essentially separates the body into the forward (anterior) half and the back (posterior) half, and is perpendicular to the median plane. Still further, it is to be understood that the transverse plane is perpendicular to both the median plane and coronal plane and is the plane which divides the body into an upper and a lower half.

The Spinal Canal and Spinal Stenosis

Referring again to FIG. 1, vertebral foramen 15 contains a portion of the ligamentum flavum 26, spinal cord 28, and an epidural space 27 between ligamentum flavum 26 and spinal cord 28 Spinal cord 28 comprises a plurality of nerves 34 surrounded by cerebrospinal fluid (CSF) contained within dural sac 32. Nerves 34 normally comprise only a small proportion of the dural sac 32 volume. Thus, CSF filled dural sac 32 is somewhat locally compressible, as localized pressure causes the CSF to flow to adjacent portions of the dural sac. Epidural space 27 is typically filled with blood vessels and fat. The posterior border of the normal epidural space 27 generally defined by the ligamentum flavum 26, which is shown in its normal, non-thickened state in FIG. 1.

FIG. 2 illustrates a case of spinal stenosis resulting from a thickened ligamentum flavum 26. Since vertebral foramen 15 is defined and surrounded by the relatively rigid bone its volume is substantially constant. Thus, thickening of ligamentum flavum 26 within vertebral foramen 15 can eventually result in compression of spinal cord 28. In particular, the thickened ligamentum flavum 26 may exert a compressive force on the posterior surface of dural sleeve 32. In addition, thickening of ligamentum flavum 26 may compress the blood vessels and fat occupying epidural space 27.

Compression of spinal cord 28, particularly in the lumbar region, may result in low back pain as well as pain or abnormal sensations in the legs. Further, compression of the blood vessels in the epidural space 27 that houses the nerves of the cauda equina may result in ischemic pain termed spinal claudication.

In order to relieve the symptoms associated with a thickened or enlarged ligamentum flavum 26, methods, techniques, and devices described herein may be employed to reduce the compressive forces exerted by the thickened ligamentum flavum on spinal cord 28 and the blood vessels in epidural space 27 (e.g., decompress spinal cord 28 and blood vessels in epidural space 27). In particular, compressive forces exerted by the thickened/enlarged ligamentum flavum 26 may be reduced by embodiments of a minimally invasive ligament decompression (MILD) procedure described herein. In some embodiments, the MILD procedure may be performed percutaneously to reduce the size of ligamentum flavum 26 by excising portions of ligamentum flavum 26. In particular; in some embodiments of the MILD procedure, the ligamentum flavum 26 is accessed, cut and removed ipsilaterally (i.e., on the same side of vertebral arch 14) by a percutaneous cranial-caudal approach. Such an embodiment of the MILD procedure may be described hereinafter as ipsilateral Approach MILD Procedure (ILAMP).

Creation of Safety Zone

As shown in FIGS. 1 and 2, ligamentum flavum 26 is posteriorly apposed to spinal cord 28. Thus, placement of tools within ligamentum flavum 26 to excise portions of ligamentum flavum 26 creates a risk of for inadvertent damage to the spinal cord 28, dural sac 32, and/or nerves 34. Thus, in preferred embodiments of the procedures described herein, prior to insertion of tissue removal tools into the ligamentum flavum 26, a gap is advantageously created between ligamentum flavum 26 and spinal cord 28 to provide a safety zone between ligamentum flavum 26 and spinal cord 28.

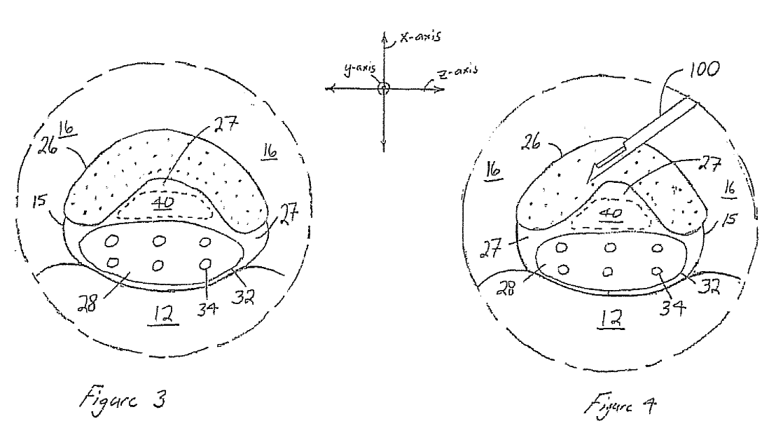

FIG. 3 illustrates an enlarged cross-sectional view of a vertebral foramen 15 within a vertebra. Vertebral foramen 15 includes epidural space 27 and spinal cord 28 containing nerves 34 and CSF within dural sac 32. Further, a thickened/enlarged ligamentum flavum 26 extends into vertebral foramen 15. To reduce the risk of damage to dural sac 32 and spinal cord 28, a safety zone 40 is created between ligamentum flavum 26 and dural sac 32.

As previously described, spinal cord 28 comprises nerves 34 surrounded by CSF and is contained within dural sac 32. Since more than 90% of the volume of dural sac 32 in the lumbar region is filled by CSF, dural sac 32 is highly compressible. Thus, even when stenosis is causing compression of spinal cord 28, in most cases it is possible to temporarily compress spinal cord 28 further. Thus, according to preferred embodiments, dural sac 32 is further compressed in the region of interest by injecting a fluid into epidural space 27 to create safety zone 40. The presence of the injected fluid comprising safety zone 40 gently applies an additional compressive force to the outer surface of dural sac 32 so that at least a portion of the CSF within dural sac 32 is forced out of dural sac 32 in the region of interest, resulting in safety zone 40 between dural sac 32 and ligamentum flavum 26.

According to some embodiments, dural sac 32 is compressed by injecting a standard radio-opaque non-ionic myelographic contrast medium or other imagable or non-imagable medium into epidural space 27 in the region of interest. This is preferably accomplished with a percutaneous injection. Sufficient injectable fluid is preferably injected to displace the CSF out of the region of interest and compress dural sac 32 to at least a desired degree. The injected medium is preferably substantially contained within the confines of epidural space 27 extending to the margins of the dural sac 32. The epidural space is substantially watertight and the fatty tissues and vascularization in epidural space 27, combined with the viscous properties of the preferred fluids, serve to substantially maintain the injected medium in the desired region of interest. This novel method for protecting spinal cord 28 column may be referred to hereinafter as “contrast-guided dural protection.”

Once a safety zone 40 has been created, a tool 100, such as the tissue excision devices and tissue retraction devices described below, may be inserted into the ligamentum flavum 26, as illustrated in FIG. 4. Tool 100 may comprise any suitable device, tool, or instrument for relieving stenosis caused by the thickened/enlarged ligamentum flavum 26 including without limitation, embodiments of tissue excision devices and tissue retraction devices described in more detail below. Further, as best illustrated in FIG. 4, tool 100 is positioned in the ligamentum flavum 26 on the opposite side of median plane 210 as tool 100 percutaneously accesses the body, such that tool 100 crosses median plane 210. In another embodiment, tool 100 is inserted and positioned in the ligamentum flavum 26 on the same side (ipsilateral) of median plane 210 as tool 100 percutaneously accesses the body, such that tool 100 does not cross median plane 210.

While it is preferred that the tip of tool 100 remain within ligamentum flavum 26 as shown, the presence of safety zone 40 reduces the likelihood that dural sac 32 will be damaged, even if the tool breaks through the anterior surface of ligamentum flavum 26.

For insertion of tool 100, a fluoroscopic window of access (FWA) is defined by the inferior margin of the lamina (contra lateral to the point of instrument entry in the soft tissues) and the dorsal margin of the contrast material that defines the epidural space. This FWA is roughly orthogonal to the long axis of the cutting instrument, which parallels the inferior surface of the lamina as in FIG. 4. The fluoroscopic plane of projection is preferably but not necessarily oriented 20-45 degrees from normal (AP projection).

Because the present techniques are preferably performed percutaneously, certain aspects of the present invention may be facilitated by imaging. In this context, the spine can be imaged using any suitable technology, including without limitation, 2D fluoroscopy, 3D fluoroscopy, CT, MRI, ultrasound or with direct visualization with fiber optic or microsurgical techniques. Stereotactic or computerized image fusion techniques are also suitable. Fluoroscopy is currently particularly well-suited to the techniques disclosed herein. Fluoroscopic equipment is safe and easy to use, readily available in most medical facilities, relatively inexpensive. In a typical procedure, using direct biplane fluoroscopic guidance and local anesthesia, epidural space 27 is accessed for injection of contrast media adjacent to the surgical site.

If the injected medium is radio-opaque, as are for example myelographic contrast media, the margins of the expanded epidural space will be readily visible using fluoroscopy or CT imaging. Thus, the safety zone created by the present contrast-guided dural compression techniques can reduce the risk of damage to the spinal cord during procedures to remove or displace portions of the ligamentum flavum and/or laminae in order to treat spinal stenosis.

Injectable Medium

If desired, the injected medium can be provided as a re-absorbable water-soluble gel, so as to better localize safety zone 40 at the site of surgery and reduce leakage of this protective layer from the vertebral/spinal canal. An injectable gel is a significant improvement on prior epidural injection techniques. The gel is preferably substantially more viscid than conventional contrast media and the relatively viscid and/or viscous gel preferably tends to remain localized at the desired site of treatment as it does not spread as much as standard liquid contrast media that are used in epidurography. This may result in more uniform compression of dural sac 32 and less leakage of contrast out of the vertebral/spinal canal. In addition, preferred embodiments of the gel are re-absorbed more slowly than conventional contrast media, allowing for better visualization during the course of the surgical procedure.

In some embodiments, a contrast agent can be included in the gel itself, so that the entire gel mass is imagable. In other embodiments, an amount of contrast can be injected first, followed by the desired amount of gel, or all amount of gel can be injected first, followed by the desired amount of contrast. In this case, the contrast agent is captured on the surface of the expanding gel mass, so that the periphery of the mass is imagable.

Any standard hydrophilic-lipophilic block copolymer (Pluronic) gel such as are known in the art would be suitable and other gels may be used as the injectable medium The gel preferably has an inert base. In certain embodiments, the gel material is liquid at ambient temperatures and can be injected through a small bore (such as a 27 gauge needle). The gel then preferably becomes viscous when warmed to body temperature after being injected. The viscosity of the gel can be adjusted through the specifics of the preparation. The gel or other fluid is preferably sufficiently viscid or viscous at body temperature to compress and protect the thecal sac in the manner described above and to remain sufficiently present in the region of interest for at least about 30 minutes. Thus, in some embodiments, the injected gel attains a viscosity that is two, three, six or even ten times that of the fluids that are typically used for epidurograms.

In certain embodiments, the injected medium undergoes a reversible change in viscosity when warmed to body temperature so that it can be injected as a low-viscosity fluid, thicken upon injection into the patient, and be returned to its low-viscosity state by cooling, In these embodiments, the injected medium is injected as desired and thickens upon warming, but can be removed by contacting it with a heat removal device, such as an aspirator that has been provided with a cooled tip. As a result of localized cooling, the gel reverts to its initial non viscous liquid state and can be easily suctioned up the cooled needle or catheter.

An example of a suitable contrast medium having the desired properties is Omnipaque® 240 available from Nycomed, New York, which is a commercially available non-ionic iodinated myelographic contrast medium. Other suitable injectable media will be known to those skilled in the art. Because of the proximity to spinal cord 28 and spinal nerves 34, it is preferred not to use ionic media in the injectable medium. The preferred compositions are reabsorbed relatively rapidly after the procedure. Thus any residual gel compression on dural sac 32 after the MILD procedure dissipates relatively quickly. For example, in preferred embodiments, the gel would have sufficient viscosity to compress dural sac 32 for thirty minutes, and sufficient degradability to be substantially reabsorbed within approximately two hours.

The injected contrast medium further may further include one or more bioactive agents. For example, medications such as those used in epidural steroid injection (e.g. Depo medrol, Celestone Soluspan) may be added to the epidural gel to speed healing and reduce inflammation, scarring and adhesions, The gel preferably releases the steroid medication slowly and prolongs the anti-inflammatory effect, which can be extremely advantageous. Local anesthetic agents may also be added to the gel, This prolongs the duration of action of local anesthetic agents in the epidural space to prolong pain relief during epidural anesthesia. In this embodiment the gel may be formulated to slow the reabsorption of the gel.

The present gels may also be used for epidural steroid injection and perineural blocks for management of acute and chronic spinal pain. Thrombin or other haemostatic agents can be added if desired, so as to reduce the risk of bleeding.

In some embodiments, the gel may also be used as a substitute for a blood patch if a CSF leak occurs. The gel may also be used as an alternative method to treat lumbar puncture complications such as post-lumbar puncture CSF leak or other causes of intracranial hypotension. Similarly, the gel may be used to patch postoperative CSF leaks or dural tears, If the dural sac were inadvertently torn or cut, then gel could immediately serve to seal the site and prevent leakage of the cerebral spinal fluid.

Percutaneous Tissue Excision

After safety zone 40 has been created, the margins of epidural space 27 are clearly demarcated by the injected medium and can be visualized radiographically if an imagable medium has been used. As mentioned above, percutaneous procedures can now safely be performed on ligamentum flavum 26 and/or surrounding tissues without injuring dural sac 32 or nerves 34 and the spinal canal can be decompressed using any of several techniques. Suitable decompression techniques include removal of tissue from the ligamentum flavum, laminectomy, laminotomy, and ligament retraction and anchoring.

In some embodiments, all or a portion of ligamentum flavum 26 and/or lamina 16 are excised using a percutaneous tissue excision device or probe 100, which may hereinafter be referred to as the MILD device, As shown schematically in FIG. 4, a device 100 may be placed parallel to the posterior and lateral margin of the safety zone 40 with its tip in the ligamentum flavum 26.

Preferred embodiments of the present tissue excision devices and techniques can take several forms. In the discussion below, the distal ends of the tools are described in detail. The construction of the proximal ends of the tools, and the means by which the various components disclosed herein are assembled and actuated, will be known and understood by those skilled in the art.

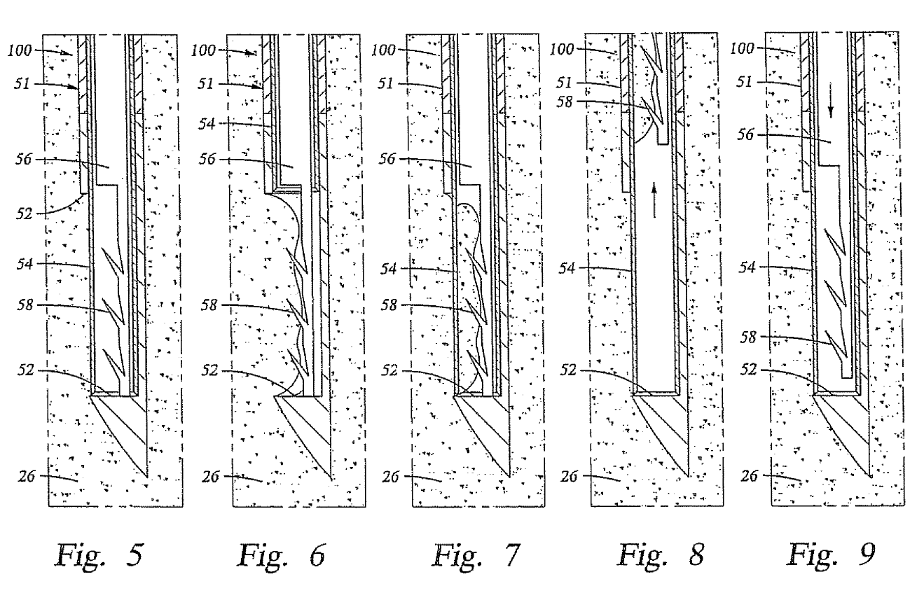

By way of example, in the embodiment shown in FIG. 4 and as illustrated in FIG. 5, device 100 may be a coaxial excision system 50 with a sharpened or blunt tip that is placed obliquely into the thickened ligamentum flavum 26 posterior to safety zone 40 under fluoroscopic guidance. The needle is preferably placed parallel to the posterior margin of the canal. Excision system 50 is preferably manufactured from stainless steel, titanium or other suitable durable biocompatible material. As shown in FIGS. 5-10, an outer needle or cannula 51 has an opening or aperture 52 on one side that is closed during insertion by an inner occluding member 54. Aperture 52 is readily visible under imaging guidance. Once needle 51 is positioned in the ligamentum flavum or other tissue removal site, inner occluding member 54 is removed or retracted so that it no longer closes aperture 52 (FIG. 6). Aperture 52 is preferably oriented away from the epidural space so as to further protect the underlying structures from injury during the surgical procedure. If it was not already present in the tool, a tissue-engaging means 56 is inserted through outer needle 51 to aperture 52 so that it contacts adjacent tissue, e.g. the ligamentum flavum, via aperture 52.

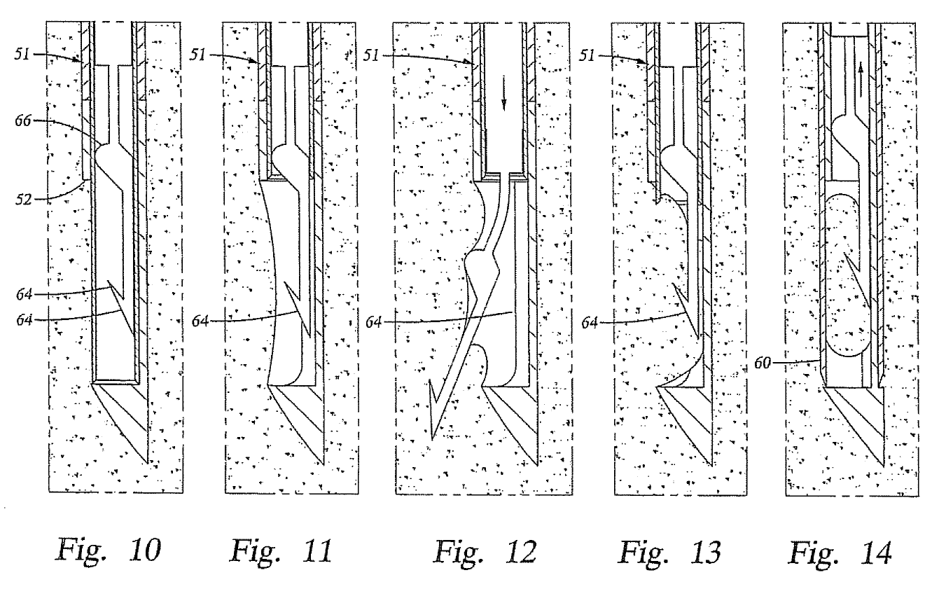

Tissue-engaging means 56 may be a needle, hook, blade, tooth or the like, and preferably has at least one flexible barb or hook 58 attached to its shaft. The barb 58 or barbs may extend around approximately 120 degrees of the circumference of the shaft. Barbs 58 are preferably directed towards the proximal end of the tool. When tissue-engaging means 56 is retracted slightly, barbs 58 allow it to engage a segment of tissue. Depending on the configuration of barbs 58, the tissue sample engaged by tissue-engaging means 56 may be generally cylindrical or approximately hemisphenical. Once needle 56 has engaged the desired tissue, inner occluding means 54, which is preferably provided with a sharpened distal edge, is advanced so that it cuts the engaged tissue section or sample loose from the surrounding tissue. Hence occluding means 54 also functions as a cutting means in this embodiments. In alternative embodiments, such as FIGS. 10-14 discussed below, a cylindrical outer cutting element 60 may extended over outer needle 51 and used in place of occluding member 54 to excise the tissue sample.

Referring still to FIGS. 5-9, once the tissue sample has been cut, tissue-engaging needle 56 can be pulled back through outer needle 51 so that the segment of tissue can be retrieved and removed from the barbs (FIG. 8). The process or engaging and resecting tissue may be repeated (FIG. 9) until the canal is adequately decompressed.

Referring briefly to FIGS. 10-14, in other embodiments, a tissue-engaging hook 64 can be used in place of needle 56 and an outer cutting member 60 can be used in place of inner occluding member 54. Hook 64 may comprise a length of wire that has been bent through at least about 270°, more preferably through 315°, and still more preferably through about 405°. Alternatively or in addition, hook 64 may comprise Nitinol™, or any other resilient metal that can withstand repeated elastic deflections, In the embodiment illustrated, hook 64 includes at least one barb 58 at its distal end. In some embodiments, hook 64 is pre-configured in a curvilinear shape and is retained within tool 100 by outer cutting member 60. When cutting member 60 is retracted, the curved shape of hook 64 urges its outer end to extend outward through aperture 52. If desired, hook 64 can be advanced toward the distal end of tool 100, causing it to extend farther into the surrounding tissue. In some embodiments, hook 64 is provided with a camming surface 66. Camming surface 66 bears on the edge of opening 52 as hook 64 is advance or retracted and thereby facilitates retraction and retention of hook 64 as it is retracted into the tool. In these embodiments, hook 64 may not extend through aperture 52 until it has been advanced sufficiently for camming surface 66 to clear the edge of the opening. Hook 64 may alternatively be used in conjunction with an inner occluding member 54 in the manner described above. As above, hook 64 can be used to retrieve the engaged tissue from the distal end of the tool.

In still other embodiments, the tissue-engaging means may comprise a hook or tooth or the like that engages tissue via aperture 52 by being rotated about the tool axis. In such embodiments (not shown) and by way of example only, the tissue-engaging means could comprise a partial cylinder that is received in outer cannula 51 and has a serrated side edge. Such a device can be rotated via a connection with the tool handle or other proximal device. As the serrated edge traverses aperture 52 tissue protruding into the tool via the aperture is engaged by the edge, whereupon it can be resected and retrieved in the manner disclosed herein.

In preferred embodiments, the working tip of tool 100 remains within the ligamentum flavum and does not penetrate the safety zone 40. Nonetheless, safety zone 40 is provided so that even an inadvertent penetration of the tool into the epidural space will not result in damage to the thecal sac. Regardless of the means by which the tissue is engaged and cut, it is preferably retrieved from the distal end of the tool so that additional tissue segments can be excised without requiring that the working tip of the tool be repositioned. A tissue-removal device such as that described below is preferably used to remove the tissue from the retrieval device between each excision.

Tissue Removal

Each piece of tissue may be removed from barbs 58 by pushing tissue-engaging means 56 through an opening that is large enough to allow passage of the flexible barbs and supporting needle but smaller than the diameter of the excised tissue mass. This pushes the tissue up onto the shaft, where it can be removed with a slicing blade or the like or by sliding the tissue over the proximal end of the needle. Alternatively, needle 56 can be removed and re-inserted into the tool for external, manual tissue removal.

It is expected that in some embodiments, approximately 8-10 cores or segments of tissue will be excised and pushed up the shaft towards the hub during the course of the procedure. Alternatively, a small blade can be used to split the tissue segment and thereby ease removal of the segment from the device. If desired, a blade for this purpose can be placed on the shaft of needle 56 proximal to the barbs.

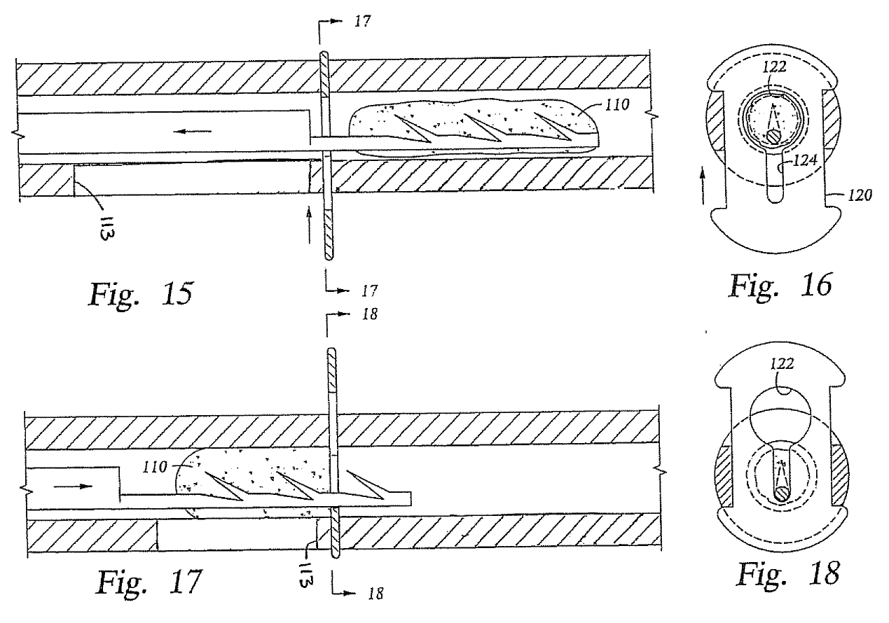

In an exemplary embodiment, shown in FIGS. 15-18, the tissue removal device may include a scraper 120 that includes a keyhole slot having a wide end 122 and a narrow end 124. To remove a tissue sample from needle 56 or hook 64, the tissue-engaging device with a mass of excised tissue 110 thereon can be retracted (pulled toward the proximal end of the tool) through wide end 122 of the slot and then re-inserted (pushed toward the distal end of the tool) through narrow end 124 of the slot. Narrow end 124 is large enough to allow passage of the barbed needle, but small enough to remove the tissue mass as the needle passes through. The removed tissue can exit the tool through an opening 113 in the tool body. By shuttling the tissue-engaging device through scraper 120 in this manner, each excised segment of tissue 110 can be removed from the device, readying the device for another excision.

In an alternative embodiment shown in FIG. 30, the tissue removal device may be constructed such that tissue is removed from the tissue-engaging device by retracting the tissue-engaging device through narrow end 124 of the slot. As above, narrow end 124 is large enough to allow passage of the shaft of the tissue-engaging device, but small enough to remove the tissue mass as the needle passes through. If the tissue-engaging device is constructed of a tough material, the barbs or the like will cut through the tissue and/or deform and release the tissue. As above, the removed tissue can exit the tool through an opening 113 in the tool body. By shuttling the tissue-engaging device through scraper 120 in this manner, each excised segment of tissue 110 can be removed from the device, readying the device for another excision.

In another alternative embodiment (not shown) an alternative mechanism for removing the tissue segment from needle 56 includes an adjustable aperture in a disc. After the tissue-bearing needle is pulled back through the aperture, the aperture is partially closed. Needle 56 and flexible hooks 58 then can pass through the partially closed aperture but the larger cylinder of tissue cannot. Thus the tissue sentient is pushed back onto the shaft. The tissue segment can either be pulled off the proximal end of the shaft or cut off of it. A small blade may be placed just proximal to the barbs to help cut the tissue segment off the shaft. The variable aperture can formed by any suitable construction, including a pair of metal plates with matching edges that each define one half of a central opening. The two pieces may be held apart by springs. The aperture may be closed by pushing the two edges together. In other embodiments, this process can be mechanically automated by using a disc or plate with an opening that is adjustable by a variety of known techniques, including a slit screw assembly or flexible gaskets.

Alternative Tissue Excision Devices

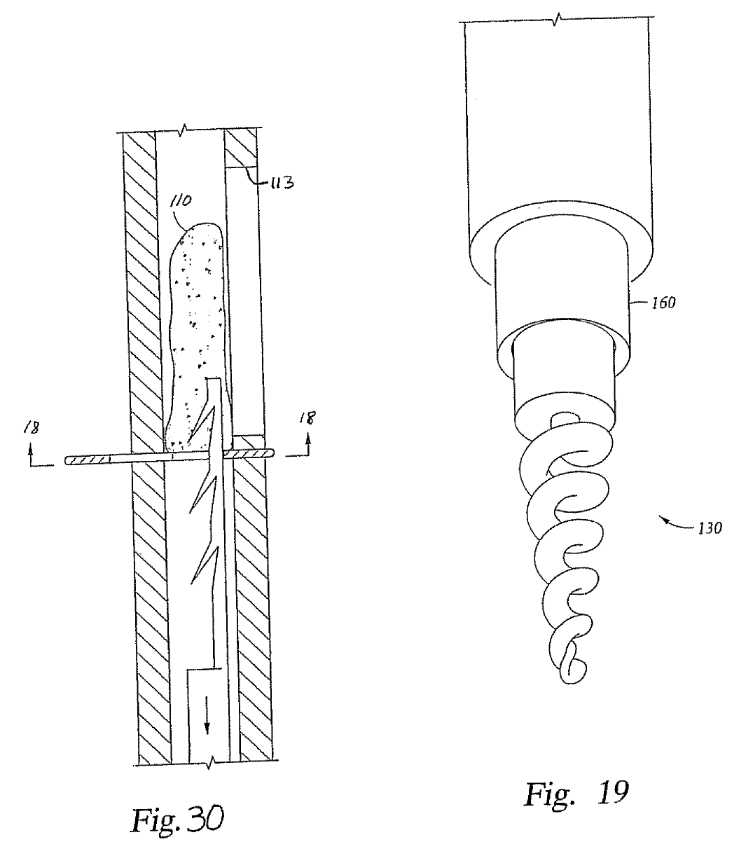

Other cutting and/or grasping devices can be used in place of the system described above. For example, embodiments of the grasping mechanism include but are not limited to: needles with flexible barbs, needles with rigid barbs, corkscrew-shaped needles, and/or retaining wires. The corkscrew-shaped needle shown in FIG. 19 works by screwing into the ligamentum flavum in the manner that a corkscrew is inserted in a cork. After the screw engages a segment of tissue, outer cutting element 60 slides over the needle, cutting a segment of tissue in a manner similar to that of the previous embodiment. In some embodiments, the cutting element can be rotated as it cuts.

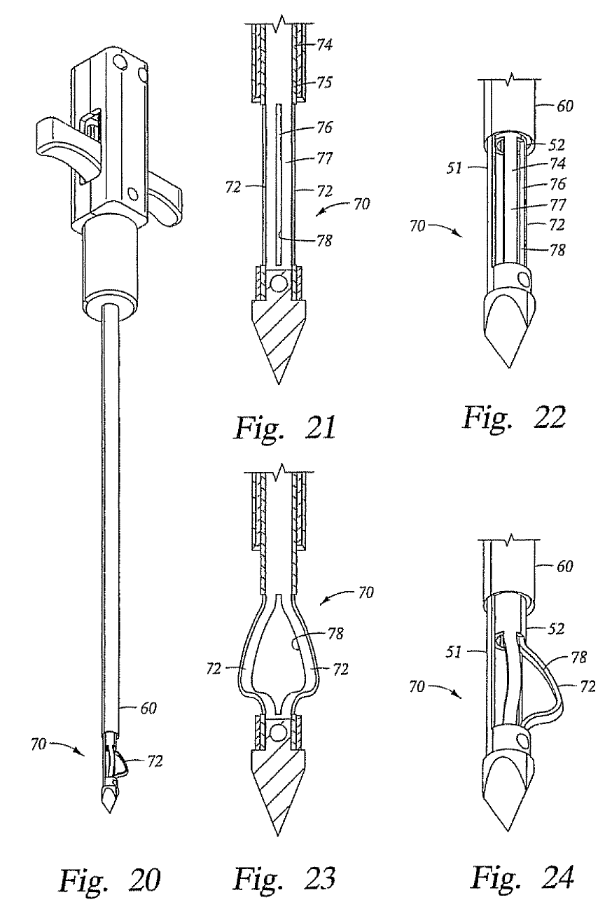

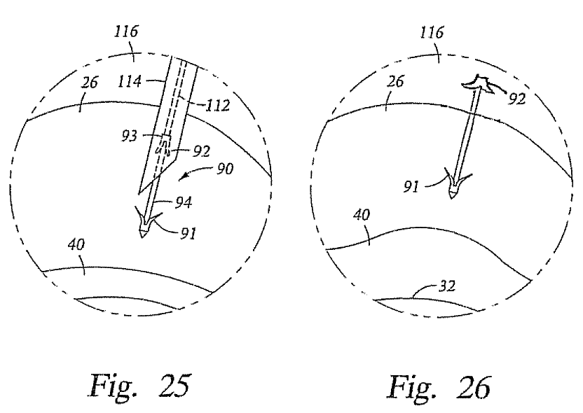

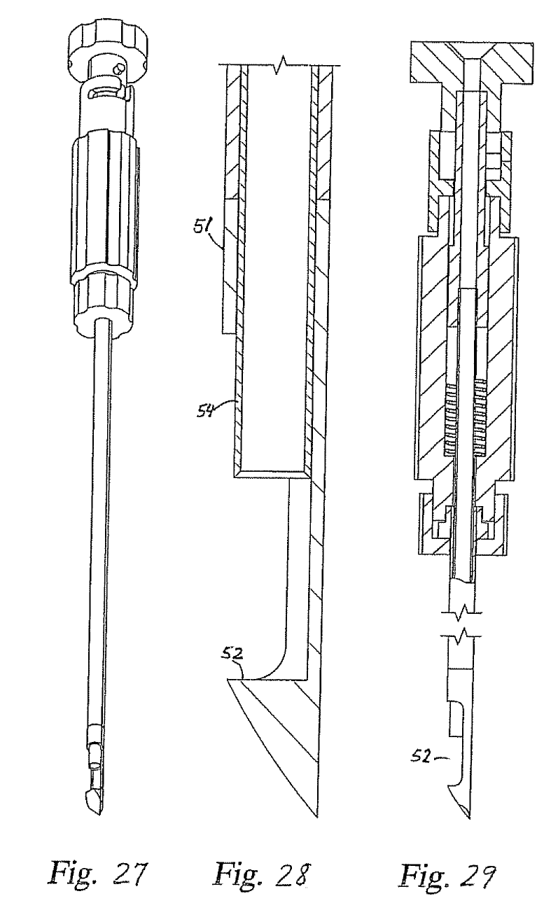

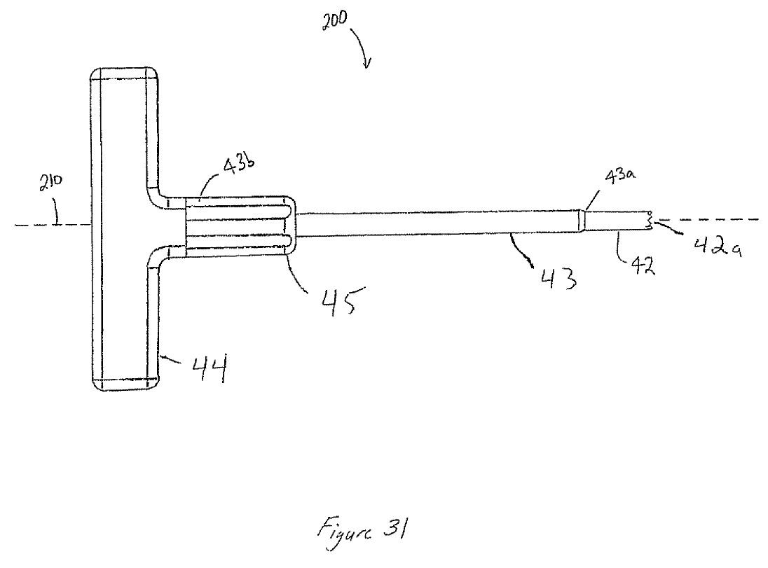

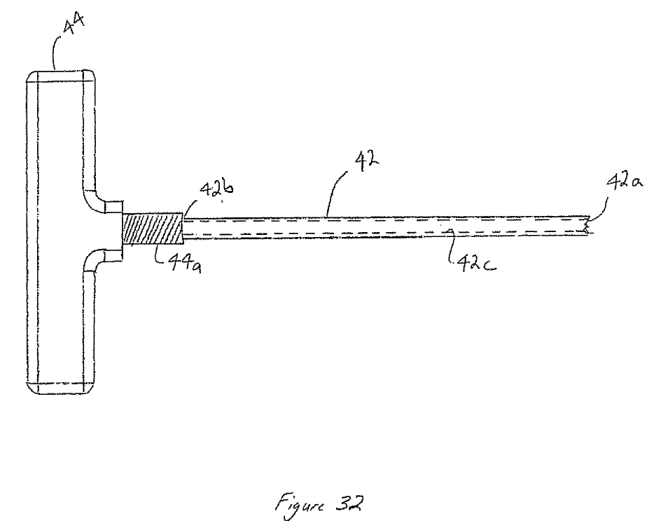

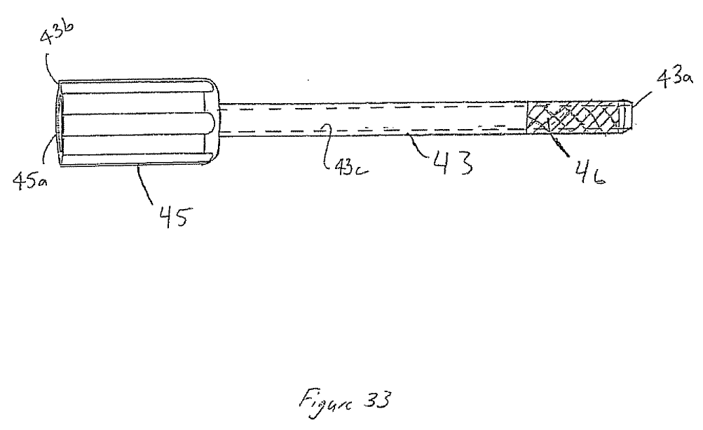

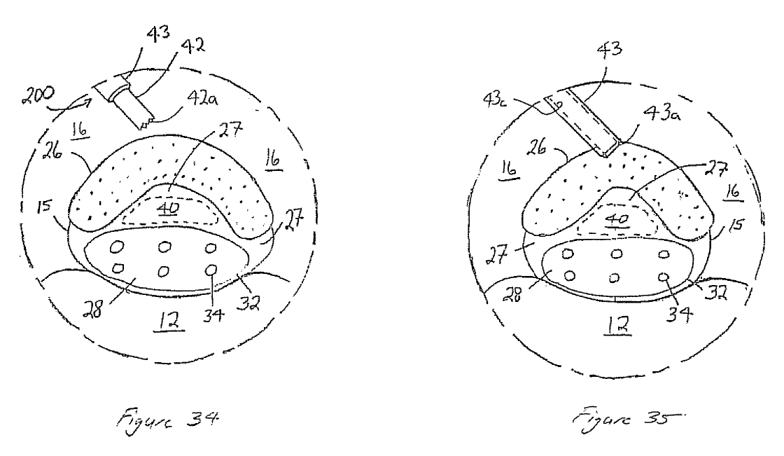

In other embodiments, shown in FIGS. 20-22, cannulated scalpel 51 houses a grasping device 70 that includes at least one pair of arcuate, closable arms 72. Closable arms 72 may be constructed in any suitable manner. One technique for creating closable arms is to provide a slotted sleeve 74, as shown. Slotted member 74 preferably comprises an elongate body 75 with at least one slot 76 that extends through its thickness but does not extend to either end of the body. Slot 76 is preferably parallel to the longitudinal axis of the sleeve. On either side of slot 76, a strip 77 is defined, with strips 77 being joined at each end of sleeve 74. It is preferred that the width of each strip 77 be relatively small. In some embodiments, it may be desirable to construct slotted member 74 from a portion of a hollow tube or from a rectangular piece that has been curved around a longitudinal axis. The inner edge of each strip that lies along slot 76 forms an opposing edge 78. The width of the piece is the total of the width of strips 77 and slot 76.

Advancing one end of sleeve 74 toward the other end of sleeve 74 causes each strip 77 to buckle or bend. If strips 77 are prevented from buckling inward or if they are predisposed to bend in the desired direction, they will bend outward, thereby forming arcuate arms 72, which extend through aperture 52 of cannulated scalpel 51, as shown in FIG. 21. As they move away from the axis of body 75, arms 72 move apart in a direction normal to the axis of body 75. Likewise, moving the ends of sleeve 74 apart causes arms 72 to straighten and to move together and inward toward the axis of the device, as shown in FIG. 22. As the arms straighten, opposing edges 78 close and a segment of tissue can be capture between them. Tissue within the grasping device may then be resected or anchored via the other mechanisms described herein.

Closable arms 72 may include on their opposing edges 78 ridges, teeth, or other means to facilitate grasping of the tissue. In other embodiments, edges 78 may be sharpened, so as to excise a segment of tissue as they close. In these embodiments, closable arms 72 may also be used in conjunction with a hook, barbed needle, pincers or the like, which can in turn be used to retrieve the excised segment from the device.

Once arms 72 have closed on the tissue, if arms 72 have not cut the tissue themselves, the tissue can be excised using a blade such as cutting element 60 above. The excised tissue can be removed from the inside of needle 51 using a tissue-engaging hook 64 or other suitable means. The process of extending and closing arms 72, excising the tissue, and removing it from the device can be repeated until a desired amount of tissue has been removed.

If desired, this cycle can be repeated without repositioning the device in the tissue. Alternatively, the tool can be rotated or repositioned as desired between excisions. It is possible to rotate or reposition the tool during an excision, but it is expected that this will not generally be preferred. Furthermore, it is expected that the steps of tissue excision and removal can be accomplished without breaching the surface of the ligament, i.e. without any part of the device entering the safety zone created by the injected fluid. Nonetheless, should the tool leave the working zone, the safety zone will reduce the risk of injury to the thecal sac.

Ligament Retraction

In some embodiments, the spinal canal may also be enlarged by retracting the ligamentum flavum, either with or without concurrent resection. Retraction is preferably but not necessarily performed after dural compression has been used to provide a safety zone. In addition, the dural compression techniques described above have the effect of pressing the ligamentum flavum back out of the spinal canal and thereby making it easier to apply a restraining means thereto.

Thus, in preferred embodiments, after a safety zone is created by epidural injection of contrast medium or gel, a retraction device 90 as shown in FIG. 23 is used to retract and compress the thickened soft tissues around the posterior aspect of the spinal canal, thereby increasing the available space for the dural sac and nerves. In the embodiment shown, retraction device 90 is a double-headed anchor that includes at least one distal retractable tissue-engaging member 91 and at least one proximal tissue-engaging member 92, each of which are supported on a body 94. Retraction device 90 is preferably constructed from an implantable, non-biodegradable material, such as titanium or stainless steel, but may alternatively be polymeric or any other suitable material. In certain preferred embodiments, body 94 is somewhat flexible. In some instances, flexibility in body 94 may facilitate the desired engagement of barbs 91, 92. Barbs 91, 92 may comprise hooks, arms, teeth, clamps, or any other device capable of selectively engaging adjacent tissue. Barbs 91, 92 may have any configuration that allows them to engage the ligamentum flavum and/or surrounding tissue, Similarly, barbs 91, 92 may be covered, sheathed, pivotable, retractable, or otherwise able to be extended from a first position in which they do not engage adjacent tissue to a second position in which they can engage adjacent tissue.

FIG. 23 shows schematically the distal and proximal retractable arms 91, 92 of a preferred ligament anchor 90. The proximal end of the anchor preferably includes a threaded connector 96 or other releasable mechanism that attaches to a support rod 100. Ligament anchor 90 may be attached to a support shaft 112 and sheathed in a guide housing 114. The distal and proximal barbs 91, 92 are prevented by guide housing 114 from engaging surrounding tissue. Housing 102 is preferably a metal or durable plastic guide housing.

The distal end of the device is preferably positioned in the ligamentum flavum under fluoroscopic guidance. If desired, an accessway through the lamina may be provided using an anchored cannula or the like. The device is held in position by support shaft 112. Distal barbs 91 are unsheathed and optionally expanded by pulling back guide housing 102, as shown in FIG. 23. Distal barbs 91 are secured in the ligamentum flavum by pulling back on the support shaft 112. With barbs 91 engaging the tissue, the ligamentum flavum is retracted posteriorly by pulling back on support shaft 112. While maintaining traction on the now-retracted ligament, proximal barbs 92 are uncovered and expanded by retracting guide housing 114, as shown in FIG. 24. Barbs 92 are preferably positioned in the soft tissues 116 in the para-spinal region so that the device is firmly anchored behind the posterior elements of the spinal canal. Once the proximal end of the anchor is engaged, support shaft 112 may be detached from body 94 as shown in FIG. 24. In this manner, the posterior margin 95 of the ligamentum flavum can be held in a retracted position, thereby expanding the canal. The procedure can then be repeated on adjacent portions of the ligamentum flavum until it is sufficiently retracted.

In an alternative embodiment, the proximal end of ligament anchor 90 may be adapted to engage the lamina. This may be accomplished by having the arm posterior to the lamina or by using the laminotomy and suturing the device to the lamina there. A knotted or knotless system or a suture plate can be used.

A second embodiment of the present method uses a plurality of retraction devices 90. In this embodiment, the retraction device is inserted through one lamina in an oblique fashion, paralleling the opposite lamina. After the distal anchor is deployed, the retraction device is pulled back and across the ligamentum flavum, thereby decompressing the opposite lateral recess of the spinal canal. This is repeated on the opposite side. This same device can also be deployed with a direct approach to the lateral recess with a curved guide housing.

While retraction device 90 is describe above as a double-headed anchor, it will be understood that other devices can be used. For example sutures, barbed sutures, staples or the like can be used to fasten the ligament in a retracted position that reduces stenosis.

Using the percutaneous methods and devices described herein, significant reductions of stenosis can be achieved. For example, a dural sac cross-sectional area less than 100 mm² or an anteroposterior (AP) dimension of the canal of less than 10-12 mm in an average male is typically considered relative spinal stenosis. A dural sac cross-sectional area less than 85 mm² in an average male is considered severe spinal stenosis. The present devices aid techniques are anticipated to cause an increase in canal area of 25 mm² per anchor or 50 mm² total. With resection and/or retraction of the ligamentum flavum, the cross-sectional area of the dural sac can be increased by 10 mm², and in some instances by as much as 20 mm² or even 30 mm² Likewise, the present invention can result in an increase of the anteroposterior dimension of the canal by 1 to 2 mm and in some instances by as much as 4 or 6 mm, The actual amount by which the cross-sectional area of the thecal sac and/or the anteroposterior dimension of the canal are increased will depend on the size and age of the patient and the degree of stenosis and can be adjusted by the degree of retraction of the ligament.

MILD

The minimally invasive ligament decompression (MILD) devices and techniques described herein allow spinal decompression to be performed percutaneously, avoiding the pain and risk associated with open surgery. Through the provision of a safety zone, the present devices and techniques offer reduced risk of spinal cord damage. In addition to improving nerve function, it is expected that decompression of the spinal canal in the manner described herein will result in improved blood flow to the neural elements by reducing the extrinsic pressure on the spinal vasculature. For these reasons, it is believed that spinal decompression performed according to the present invention will be preferable to decompression operations performed using currently known techniques.

Dural Shield

In some embodiments (not shown), a mechanical device such as a balloon or mechanical shield can also be used to create a protective guard or barrier between the borders of the epidural space and the adjacent structures. In one embodiment a durable expandable device is attached to the outside of the percutaneous laminectomy device, preferably on the side opposite the cutting aperture. The cutting device is inserted into the ligamentum flavum with the expandable device deflated. With the aperture directed away from the spinal canal, the expandable device is gently expanded via mechanical means or inflated with air or another sterile fluid, such as saline solution, via a lumen that may be within or adjacent to the body of the device. This pushes the adjacent vital structures clear from the cutting aperture of the device and simultaneously presses the cutting aperture into the ligament. As above, the grasping and cutting needles can then be deployed and operated as desired. The balloon does not interfere with tissue excision because it is located on the side opposite the cutting aperture. The cutting needle may be hemispherical (semi-tubular) in shape with either a straight cutting or a sawing/reciprocating blade or may be sized to be placed within the outer housing that separates the balloon from the cutting aperture.

In another embodiment, a self-expanding metal mesh is positioned percutaneously in the epidural space. First the epidural space is accessed in the usual fashion. Then a guide catheter is placed in the epidural space at the site of the intended surgical procedure. The mesh is preferably compressed within a guide catheter, When the outer cover of the guide catheter is retracted, the mesh expands in the epidural space, protecting and displacing the adjacent dural sheaths. At the conclusion of the surgical procedure, the mesh is pulled back into the guide sheath and the assembly removed. The mesh is deformable and compresses as it is pulled back into the guide catheter, in a manner similar to a self-expanding mesh stent. There are many commercially available self-expanding stents approved and in use in other applications. However, using a self-expandable mesh as a device within the epidural space to protect and displace the thecal sac is novel.

Anchoring Laminotomy Portal (ALP) Device

In some MILD procedures or other percutaneous surgical procedures, it may be desirable to use bone adjacent the surgical site as an anchor for various surgical tools and devices. For instance, when performing a ligament decompression as described above, it may be desirable to anchor the tissue-excision tool (e.g., tool 100) in the lamina of an adjacent vertebra. Anchoring provides a relatively firm base for the tools and devices used in the procedure, thereby offering the potential for more stable and consistent procedures. Thus, according to certain embodiments of the MILD procedure described herein, an image-guided percutaneous lumbar laminotomy is performed with the use of an anchoring laminotomy portal (ALP) device. The ALP is anchored to bone and provides access to the underlying tissues, ligaments, fat, and epidural space. For example, in some embodiments, the ALP may comprise a cannula having an inner bore that provides percutaneous access to the underlying ligamentum flavum in the region of interest. In such embodiments, the laminotomy is performed without disrupting the continuity of the entire lamina while accessing intervertebral discs or neural structures.

In contrast, according to some conventional techniques for reducing stenosis, an incision is made in the back and the muscles and supporting structures are stripped away from the spine, exposing the posterior aspect of the vertebral column. The thickened ligamentum flavum is then exposed by removal of a portion of the vertebral arch, often at the laminae, covering the back of the spinal canal (laminectomy). The thickened ligamentum flavum ligament can then be excised by sharp dissection with a scalpel or punching instruments However, as previously described, this approach typically requires general anesthesia and often results in a lengthy hospital stay and a painful and lengthy recovery

By contrast, the ALP device, described in more detail below, allows a surgeon to achieve percutaneously access the ligamentum flavum without cutting a large hole in the tissue or stripping tissue, muscle, or ligaments from the lamina and without performing a laminectomy. While the invention is described below in terms of a laminotomy, it will be understood that it is equally applicable to any other operation in which an adjacent bone is used as an anchoring base.

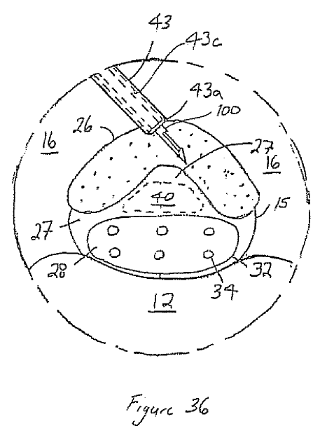

Referring now to FIG. 31, a tool 200 for installing an ALP is illustrated. In general, tool 200 is used to install an ALP through the bone of a lamina in order to provide percutaneous access to, and decompress, an enlarged ligamentum flavum. Tool 200 comprises a bone-cutting member 42, a portal 43, a portal access or cup 45, and a handle 44. FIG. 31 shows tool 200 in its assembled configuration, prior to installation and anchoring of portal 43 to a bone. In the assembled configuration, bone-cutting member 42 and portal 43 are concentric and coaxial with each other, sharing the same longitudinal axis 210.

Referring to FIG. 32, bone-cutting member 42 is shown separated from portal 43. Bone-cutting member 42 preferably comprises a cannulated bone saw. In the embodiment illustrated in FIG. 32, bone-cutting member 42 includes a free or cutting end 42a and a handle end 42b coupled to handle 44. Bone cutting end 42a includes a sharpened, serrated edge configured to saw through bone. Handle end 42b of bone-cutting member 43 may be fixed or releasably attached to handle 44. Bone-cutting member 42 also includes a central bore 42c that accommodates bone cut by cutting end 42a as bone-cutting member 42 is advanced through bone.

Referring now to FIG. 33, portal 43 is shown separated from bone-cutting member 42 and handle 44. Portal 43 preferably comprises a cannula. In the embodiment illustrated in FIG. 33, portal 43 has a first end 43a, a second end 43b, and a through bore 43c extending therebetween. The length of portal 43 measured between first end 43a and second end 43b is preferably in the range of 2 to 6 inches long.

First end 43a preferably includes a sharpened, beveled outer surface that enables first end 43a, and hence portal 43, to be more easily advanced through a hole in bone cut by bone-cutting member 42. As will de described in more detail below, when portal 43 is anchored in bone to perform a surgical procedure, first end 43a is positioned adjacent, or in, the region of interest such that bore 43c provides access through portal 43 to the region of interest. Portal 43 preferably comprises a cannula.

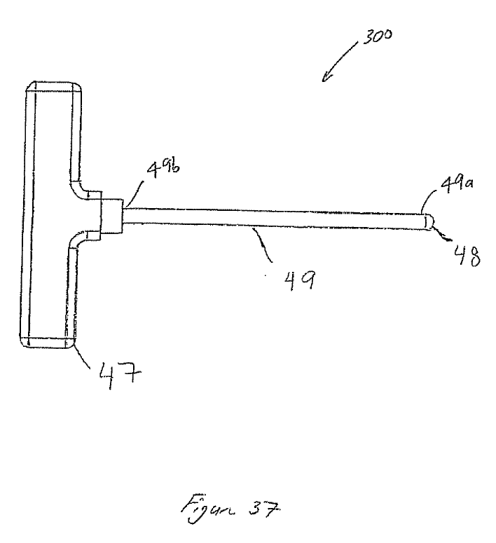

Second end 43b of portal 43 includes a portal access or cup 45 generally coaxial with portal 43. Portal cup 45 facilitates insertion of tools into through bore 43c of portal 43. In addition, portal cup 45 is preferably releasably affixed to handle 44, such as by threads or the like. For instance, portal cup 45 may include threads on its inner surface that mate with threads 44a (FIG. 32) of handle 44, thereby releasably coupling portal cup 45, and hence portal 43, with handle 44 and bone-cutting member 43. U.S. application Ser. No. 11/426,340, which is hereby incorporated herein by reference in its entirety, discloses alternative releasable couplings that may be used to releasably connect portal cup 45 and handle 44 and/or couple handle 44 and bone-cutting member 42 When portal 43 is coupled to handle 44, portal 43 is restricted from moving axially relative to bone-cutting member 43. However, once portal 43 is released or de-coupled from handle 44, portal 43 is free to move axially relative to bone-cutting member 42. Thus, tool 200 and/or portal 43 may be described as having a first position with portal 43 releasably coupled to handle 44 (FIGS. 31 and 34), and a second position with portal 43 de-coupled or released from handle 44 and bone-cutting member 42 (FIGS. 32, 33, 35, and 36).

Alternatively, portal 43 may include threads on its inner surface (not shown) adapted to mate with threads provided on the outer surface of bone-cutting member 42. In such embodiments, portal 43 will move axially relative to bone-cutting member 42 as the two components are unthreaded. Further, once completely unthreaded, portal 43 is free to move axially relative to bone-cutting member 42.

Referring still to FIG. 33, as previously described, first end 43a of portal 43 preferably includes a bevel or sharpened edge. In addition, portal 43 preferably includes ridges, knurling or other surface features 46 on its outer surface proximal first end 43a that enhance engagement between portal 43 and the surrounding bone to which it is anchored. Surface features 46 essentially increase the frictional forces between portal 43 and the surrounding bone, thereby increasing the stability of the engagement therebetween and reducing the potential for inadvertent movement of portal 43 relative to the surrounding bone during the procedure.

Referring now to FIGS. 34-36, selected views of tool 200 installing an ALP (e.g., portal 43) in lamina 16 to provide access to thickened ligamentum flavum 26 (e.g., region of interest) are illustrated. As shown in FIG. 34, in its assembled configuration, bone-cutting member 42 of tool 200 is employed to bore a hole through lamina 16. In some embodiments, handle 44 may be rotated about axis 210 to enable cutting end 42a of bone-cutting member 42 to cut and advance through lamina 16. After a hole has been cut through lamina 16, or while the hole is being cut, portal 43 is released from handle 44 and advanced through the bone by means threadingly disengaging portal 43 with handle 44 (if portal cup 45 is threaded to handle 44) and/or threaded disengagement of portal 43 with bone-cutter member 42 (if the inner surface of portal 43 is threaded to the outer surface of bone-cutting member 42). Portal 43 is advanced relative to bone-cutting member 42 through the hole created in lamina 16 by hone-cutter 42. Portal 43 is preferably advanced until first end 43a of portal 43 is positioned adjacent to, or just within, the thickened ligamentum flavum 26 in the region of interest as best shown in FIG. 34.

It should be appreciated that since bone-cutting member 42 is sized to fit within bore 43c of portal 43, the outside diameter of portal 43 is slightly larger than the outside diameter of bone-cutting member 42. Thus, the outside diameter of portal 43 will be slightly larger than the hole created by bone-cutting member 43, resulting in an interference fit between portal 43 and the surrounding lamina 16. In addition, as previously described, textured surface features 46 shown in FIG. 33 further enhance the engagement between portal 43 and lamina 16.

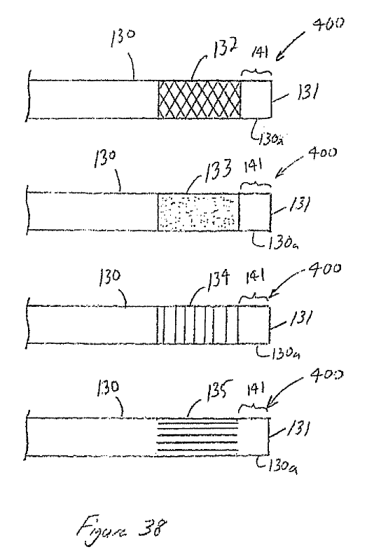

Once portal 43 is sufficiently positioned, bone-cutting member 42 can be completely removed and separated from bore 43c of portal 43. Once bore 43c is completely cleared of bone-cutting member 42, bore 43c provides a percutaneous passageway to the thickened ligamentum flavum 26, as best shown in FIG. 35. For instance, bore 43c of portal 43 provides access to tool 100 previously described so that tool 100 can cut and remove portions of thickened ligamentum flavum 26, thereby decompressing ligamentum flavum 26, as shown in FIG. 36. Although bone-cutting member 42 and portal 43 are described herein as distinct components that may be coupled together (FIG. 31) or separated (FIGS. 32 and 33), in other embodiments, bone-cutting member 42 and portal 43 may be a single integral component, such that portal 43 is in place once at the same time as the hole is being created.

As previously described, portal 43 is held in place by friction resulting from the interference fit between portal 43 and lamina 16, as well as from the friction resulting from engagement of textured surface features 46 of portal 43 and lamina 16. Thus, portal 43 is relatively stable once anchored to lamina 16, thereby ensuring proper placement of first end 43a adjacent, or in, the thickened ligamentum flavum 26 during the procedure. Thus, other tools (e.g., tool 100) inserted through bore 43c of portal 43 and into the ligamentum flavum 26 on the other side of lamina 16 can be consistently and reliably emplaced in a desired spot in the ligamentum flavum 26. Since portal 43 is held in place and anchored to the bone (e.g., lamina 16) through which it passes, portal 43 allows repeated access to a desired tissue site (e.g., ligamentum flavum 26).

Referring now to FIG. 37, a repositioning and removal device 300 is illustrated. Device 300 is employed to reposition portal 43 as necessary, and remove portal 43 from the bone through which it is anchored, once percutaneous access via portal 43 is no longer needed. Thus, device 300 is preferably included with or as part of ALP installation tool 200 previously described.

Repositioning and removal device 300 includes a handle 47 and an elongate rigid rod or body 49 extending from handle 47. Body 49 has a free end 49a and a fixed end 49b. Free end 49a preferably includes a rounded or blunt tip 48, while fixed end 49b is fixed to handle 47, such that body 49 does not move rotationally or translationally relative to handle 47. In addition, body 49 comprises a rigid tube or rod having an outside diameter that is approximately equal to or slightly smaller than the diameter of bore 43c of portal 43. Thus, elongate body 49 may be disposed coaxially within bore 43c of portal 43.

As previously discussed, device 300 is utilized to reposition portal 43, if necessary, after portal 43 is installed and anchored. Further, device 300 can be used to remove portal 43 once access through bore 43c to a region of interest is no longer needed (e.g., the procedure is complete). For instance, if portal 43 has been installed and it is desired to reposition it, body 49 of repositioning and removal device 300 may be inserted into bore 43c portal 43 and used to provide leverage and control as portal 43 is repositioned. It is expected that movement of body 29 through at least 10 degrees, more preferably through at least 20 degrees, and still more preferably through at least 30 degrees will be possible while retaining the advantages of the anchored portal 43.

During repositioning and/or removal of portal 43, rigid body 49 disposed within bore 43c also helps prevent portal 43 from buckling or bending during such movement. In addition, blunt tip 48 on first end 49a of body 49 ensures that tissue and nerves near free end 49a of body 49 and/or near first end 43a of portal 43 will not be damaged during the repositioning process

To remove portal 43, repositioning and removal device 300 is coupled to portal 43 such that portal 43 and device 300 are restricted from moving translationally relative to each other. Then device 300, along with portal 43, is pulled from the patient. Once device 300 is coupled to portal 43, slight back-and-forth repositioning of portal 43 may be necessary to loosen the engagement between portal 43 and the bone to which it is anchored, thereby enabling portal 43 to be more easily removed.

Device 300 and portal 43 may be coupled by any suitable manner including without limitation mating threads or the like. For instance, handle 47 may be provided with external threads similar to threads 44a (FIG. 32) that can engage mating threads provided on the inner surface of portal cup 45 and/or bore 43c of portal 43. Alternatively, any other suitable means may be used for removing portal 43 from the bone.

The components of ALP tool 200 and repositioning and removal device 300 may be constructed of conventional materials suitable for surgical instruments. By way of example only, portal 43 can comprise 400 series stainless steel, 17 series stainless steel, 300 series stainless steel, or any other suitable material. Handles 44, 47 can be connected to bone-cutting member 42 and body 49, respectively, by over-molding, press fitting, adhesives, or combinations thereof.

While ALP tool 200 is described in the foregoing descriptions in terms of a laminotomy, and portal 43 is described as being anchored in a lamina 16, it should be appreciated that the present anchoring methods and devices can be used in any situation where it is desired to pierce a bone and perform a surgical operation using tools emplaced through the resulting opening. The anchoring portal is particularly useful when it is desired to perform a repeated operation through an opening in a bone, as the anchoring portal is fixed relative to the bone and ensures that the tool will be inserted along the same axis each time the portal is used. Similarly, the present devices can be used to adjust the angle of access in a controlled manner.

Bone Wax Application Device

Using ALP tool 200 as previously described, percutaneous access to a thickened ligamentum flavum 26 via portal 43 is provided. In particular, a hole is cut through lamina 16 by bone-cutting member 42 and portal 43 is positioned and anchored therethrough. However, in some instances, cutting of bone (e.g., lamina 16) may cause undesirable bone bleeding. Since some conventional methods to stop bleeding (e.g., cauterization, application of pressure, etc.) may be insufficient and/or ineffective to reduce or prevent bone bleeding, in preferred embodiments of the invention described herein, bone wax may be applied to the inner surface of the bore or hole cut through the bone. In general, the application of bone wax creates a physical barrier that plugs vascular openings in the bone, thereby reducing bone bleeding during and after the procedure in which the bone is cut.

FIG. 38 depicts four embodiments of a bone wax application device 400. Device 400 is useful in promoting bone hemostasis by applying bone wax at the site of bone trauma during and/or after MILD procedures employing ALP tool 200 described above. Preferred embodiments of bone wax application device 400 comprise an elongate hollow body 130 having a textured surface treatment 132, 133, 134, 135 proximal a free end 130a, but not extending all the way to the tip 131 of free end 130a. Textured surface treatment 132, 133, 134, 135 provides surface irregularities and roughness that accommodate the bone wax, engage the bone wax, and hold the bone wax in place during application of the bone wax to the cut bone. A portion 141 of the outer surface between textured surface treatment 132, 133, 134, 135 and tip 131 is smooth and forms a band ensure that the bone wax does not go beyond the intended application site. Portion 141 preferably spans a distance such as 1/16, ⅛, ¼ or ½ inch from tip 131.

In alternative embodiments, the entire hollow body 130 may comprise a surface treatment. Examples of suitable surface treatments include diamond knurling 132, sand blasting 133, machined transverse grooves 134, or machined longitudinal grooves 135. Other surface treatments may be created using media blasting, plasma etching, bead blasting, or any other suitable technique. In still other embodiments, the distal portion of bone wax application device 400 is tapered, so that the diameter of the device increases in the proximal direction. This helps the device apply radial pressure to the bone wax as it is advanced.

Hollow body 130 may comprise any suitable device including without limitation a surgical cannula, a catheter, a Hypotube, portal 43 previously described, bone-cutting member 43 previously described, or the like. For instance, surface feature 46 on portal 43 (FIG. 33), which engages the bone through which it passes, may be used as a textured surface to apply bone wax to the cut bone within which it is disposed. As another example, a textured surface treatment may be provided neat cutting end 42a of bone-cutting member 42 previously described to apply bone wax to the cut bone as bone-cutting member 42 cuts and advances through the bone.

The present bone wax application device maybe made of any materials conventionally used in surgical instruments. Such materials include 400 series stainless steel, 17 series stainless steel, or 300 series stainless steel. The invention may be fabricated by any means including machining, laser-cutting, electro-mechanical deposition, and electro-polishing.

While preferred embodiments of this invention have been shown and described, modifications thereof can be made by one skilled in the art without departing from the scope or teaching of this invention. For example, the means by which the safety zone is formed may be varied, the shape and configuration of the tissue excision devices may be varied, and the steps used in carrying out the technique may be modified. Accordingly, the invention is not limited to the embodiments described herein, but is only limited by the claims that follow, the scope of which shall include all equivalents of the subject matter of the claims. Likewise, the sequential recitation of steps in a claim, unless explicitly so stated, is not intended to require that the steps be performed in any particular order or that a particular step be completed before commencement of another step.

What is claimed is:

1. A device for providing percutaneous access to a surgical site, comprising:
a handle;
a bone-cutting member extending from said handle, wherein said bone-cutting member includes a handle end fixed to said handle and a cutting end;
a portal including a first end, a second end, and a through bore extending therebetween, wherein said bone-cutting member is disposed within said through bore;
wherein said portal has a first position in which said second end is releasably coupled to said handle and a second position in which said second end is released from said handle and said bone-cutting member.

2. The device according to claim 1 wherein said portal threadingly engages said handle.

3. The device according to claim 1 wherein said bone-cutting member comprises a cannulated bone saw.

4. The device according to claim 1 wherein said first end of said portal includes a beveled edge.

5. The device according to claim 1 wherein said second end of said portal includes a portal cup adapted to releasably engage said handle.

6. The device of claim 1 wherein said portal has an outer surface and wherein at least a portion of said outer surface includes a textured surface feature proximal said first end.

7. The device of claim 6 wherein said textured surface feature is spaced at least 1/16^{th} of an inch from said first end.

8. The device of claim 7 wherein said textured surface feature is spaced at least ⅛^{th} of an inch from said first end.

9. The device of claim 6 wherein said textured surface feature is adapted to hold and apply bone wax to a bone.

10. The device of claim 6 wherein said textured surface feature comprises diamond knurling, sand blasting, bead blasting, media blasting, vertical grooves, horizontal grooves or plasma etching.

11. The device of claim 1 wherein said portal has a length measured from said first end to said second end, wherein said length of said portal is between 2 and 6 inches.

12. The device of claim 1 further comprising a repositioning device including a rigid elongate rod having an outside diameter that is less than the inside diameter of said through bore of said portal.

13. The device of claim 1 wherein said portal comprises 400 series stainless steel, 17 series stainless steel, or 300 series stainless steel.

14. A system for performing a percutaneous ligamentum flavum decompression comprising:
a handle;
a bone-cutting member extending from said handle;
a portal comprising a cannulated member extending from said handle and concentric with said bone-cutting member, wherein said portal is releasably coupled to said handle;
a tissue-excision device sized and configured to pass through said portal.

15. The system of claim 14 wherein said portal includes a first end, a second end, and a through bore extending therebetween, wherein said bone-cutting member is disposed within said through bore.

16. The system of claim 15 wherein said portal has a first position with said second end releasably coupled to said handle and a second position with said second end released from said handle and said bone-cutting member.

17. The system of claim 14 wherein said bone-cutting member comprises a cannulated bone saw.

18. The device according to claim 9 wherein said portal and said bone-cutting member are formed as a single integral component.

19. The device according to claim 14 wherein said second end of said portal threadingly engages said handle.

20. A method for treating stenosis in a spine, the spine including a thecal sac, a spinal canal and an epidural space therebetween, the stenosis determining a region of interest in the spine, comprising the steps of:
a) compressing the thecal sac in the region of interest by injecting a fluid to form a safety zone and establish a working zone, the safety zone lying between the working zone and the thecal sac;
b) percutaneously cutting a hole through a lamina of the spine adjacent the region of interest;
c) positioning a portal through said hole to provide access to the region of interest;
d) inserting a tissue-excision tool through the portal and into tissue in the working zone;
e) using the tool to percutaneously reduce the stenosis; and
f) utilizing imaging to visualize the position of the tool during at least a part of step e).

21. The method of claim 21 further comprising the step of applying bone wax to said hole cut through the lamina to reduce bone bleeding.
